(19) United States
(12) Patent Application Publication
(10) Pub. No.: US 2016/0257634 A1
(43) Pub. Date: Sep. 8, 2016

(54) PREPARATION OF 2,2'-BIARYLS IN THE PRESENCE OF MOLYBDENUM(V) CHLORIDE

(71) Applicant: Evonik Degussa GmbH, Essen (DE)

(72) Inventors: Katrin Marie Dyballa, Recklinghausen (DE); Robert Franke, Marl (DE); Dirk Fridag, Haltern am See (DE); Michael Mirion, Mainz (DE); Thomas Quell, Mainz (DE); Siegfried R. Waldvogel, Gau-Algesheim (DE)

(21) Appl. No.: 15/058,426

(22) Filed: Mar. 2, 2016

(30) Foreign Application Priority Data
Mar. 5, 2015 (DE) 10 2015 203 967.6

(51) Int. Cl. C07C 41/30; C07C 37/14; C07C 391/02

(52) U.S. Cl. CPC C07C 41/30; C07C 391/02; C07C 37/14

(57) ABSTRACT

The invention is concerned with a process for preparing 2,2′-biaryls, especially of 2,2′-biphenols in which a reaction mixture comprising a first aryl, a second aryl and selenium dioxide is heated, the first aryl being reacted with the second aryl to give the corresponding 2,2′-biaryl, wherein the reaction mixture additionally comprises molybdenum(V) chloride.

### RELATED APPLICATION DATA

The present application hereby claims priority to German Application No. DE 10 2015 203 967.6 filed Mar. 5, 2015, which is incorporated herein by reference in its entirety.

### FIELD

The invention is concerned with the preparation of 2,2′-biaryls, especially of 2,2′-biphenols. The starting substances used are either two aryls or two phenols or a 2,2′-selenobiaryl ether.

### BACKGROUND OF INVENTION 2,2′-Biaryl refers to compounds in which two aryl groups are joined to one another via a single bond. The simplest biaryl is biphenyl. It is shown in Scheme 0.

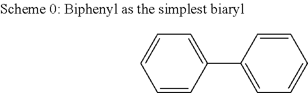 [SMILES: c1ccc(-c2ccccc2)cc1]

A 2,2′-biphenol is a 2,2′-biaryl substituted by at least two OH groups, and these are also referred to as 2,2′-dihydroxybiaryls.

Biphenols and biaryls serve as synthesis units for catalytically active substances and are therefore of industrial interest. Biaryls are important synthesis units for liquid crystals, organic devices, dyes, ligands for metal catalysts, and find uses even in medical areas, since these structures are ubiquitous in biologically active, naturally occurring products (cf. R. Noyori, Chem. Soc. Rev. 1989, 18, 187 and I. Cepanec, Synthesis of Biaryls, Elsevier, New York, 2004). The 2,2′-biphenols in particular can be used for the purpose; cf. WO 2005/042547. These are employed particularly as ligand components for catalysts. In this case, the biphenol can be used, for example, as ligand unit in the enantioselective catalysis (cf. Y. Chen, S. Yekta, A. K. Yudin, Chem. Rev. 2003, 103, 3155-3211; J. M. Brunel Chem. Rev. 2005, 105, 857-898; S. Kobayashi, Y. Mori, J. S. Fossey, Chem. Rev. 2011, 11, 2626-2704).

Biaryls and biphenols can respectively be prepared by direct coupling of aryls and phenols to give corresponding biaryl or biphenol derivatives.

The umbrella terms “aryls” and “phenols” should be understood in this connection to mean both unsubstituted and substituted compounds. Substitution here is on the benzene ring.

The coupling of aryls or phenols to give the corresponding biaryl or biphenol derivatives constitutes a major challenge, since these reactions are often neither regio- nor chemoselective.

One possible way of synthesizing these biphenols is by means of electrochemical processes. In this context, carbon electrodes such as graphite, glassy carbon, boron-doped diamond or noble metals such as platinum were used; cf. WO2010139687A1 and WO2010023258A1. A disadvantage of these electrochemical methods is the cost of some of the apparatus, which has to be manufactured specially. Moreover, scale-up to the ton scale, as is typically required in industry, is sometimes very complex and in some cases even impossible.

Direct cross-coupling of unprotected phenol derivatives under conventional organic conditions has been possible only in a few examples to date. For this purpose, usually superstoichiometric amounts of inorganic oxidizing agents such as AlCl₃, FeCl₃, MnO₂, or 2,3-dichloro-5,6-dicyano-1,4-benzoquinone, which is organic, have been used; cf. G. Sartori, R. Maggi, F. Bigi, M. Grandi, J. Org. Chem. 1993, 58, 7271.

Alternatively, such coupling reactions are conducted in a multistage sequence. In this case, leaving functionalities and often toxic, complicated transition metal catalysts based on palladium, for example, are used; cf. L. Ackermann, Modern Arylation Methods, Wiley-VCH, Weinheim, 2009, X. Chen, K. M. Engle, D.-H. Wang, J.-Q. Yu, Angew. Chem. Int. Ed. 2009, 48, 5094-511, I. V. Seregin, V. Gevorgyan, Chem. Soc. Rev. 2007, 36, 1173-1193, G. Dyker, Handbook of C-H Transformations, Wiley-VCH, Weinheim, 2005.

A great disadvantage of the abovementioned methods for phenol coupling is the need for dry solvents and for exclusion of air. Both mean a high level of complexity, specifically when the process is to be used on the industrial scale.

Furthermore, the reactions described in the prior art often give rise to toxic by-products which have to be removed from the desired product in a complex manner and disposed of at great cost. The increasing scarcity of raw materials (for example boron and bromine) and the rising relevance of environmental protection are increasing the cost of such transformations. Particularly in the case of utilization of multistage syntheses, an exchange of various solvents is necessary, which constitutes a high level of complexity and is an additional cost factor.

The problem of the cost-driving necessity of the exclusion of air has already been solved by the use of selenium dioxide as oxidizing agent:

For instance, DE102014209967A1 of May 26, 2014, which was yet to be published at the filing date, describes the preparation of 2,2′-biphenols using selenium dioxide in the presence of an acid. A disadvantage of this route is that selenobiaryl ethers may also form as secondary components according to the reaction conditions. These may then have to be removed in a costly and inconvenient manner, which of course makes the process much more expensive in an industrial scale implementation.

The likewise as yet unpublished DE102014209976A1 from the same date shows that it is possible to use a fluorinated solvent in place of the acid in order to couple two phenols without exclusion of air to give corresponding biphenols. However, fluorinated solvents are particularly difficult to handle, especially in the case of industrial scale use, since hydrofluoric acid may form according to the conditions. This is very difficult indeed for safety reasons. An additional factor is that the preparation of the fluorinated solvents is complex and the use thereof is correspondingly costly.

In the light of this prior art, the problem addressed by the invention was that of specifying a process for preparing 2,2′-biaryls, especially of 2,2′-biphenols, by coupling corresponding aryls or phenols, which can be conducted on the industrial scale. This especially means that it is thus possible to prepare the desired substances selectively on the ton scale, without having to handle and/or dispose of toxic substances to an undue extent. Furthermore, a minimum amount of useless by-products, for instance selenobiaryl ethers, should arise. The process should also conserve resources and therefore if at all possible work without bromine and boron. The use of fluorinated solvents should also be avoided. Finally, it is additionally desirable to be able to work without exclusion of air.

This problem was additionally solved by conducting the coupling of the aryls/phenols in the presence of selenium dioxide, but additionally with the addition of molybdenum(V) chloride.

This is because it has been found that the presence of molybdenum (V) chloride alongside selenium dioxide in the reaction mixture surprisingly makes it possible to dispense with the presence of a fluorinated solvent or of an acid. Due to the molybdenum (V) chloride, it is also possible to conduct the process in a bromine- and boron-free manner. This is impossible, for example, in the case of conventional palladium-catalyzed couplings, since it is necessary to use bromine- or boron-containing leaving functionalities inter alia therein. Otherwise, it would not be possible to conduct the reaction.

Molybdenum (V) chloride appears to act as a second oxidizing agent alongside the selenium dioxide, possibly even additionally in a catalytic manner. Although it is consumed in the reaction, it is not incorporated into the biaryls or phenols. Consequently, it is required only in small amounts. Since molybdenum (V) chloride (MoCl₅) is itself biocompatible and does not form any toxic by-products in the reaction system either, the advantages of the additional use of MoCl₅ predominate.

A problem with the use of selenium dioxide is that the corresponding 2,2′-selenobiaryl ether and the corresponding Pummerer ketone can be obtained as by-products in large amounts. In the case of an unfavorable reaction regime, it may even be the case that the 2,2′-selenobiaryl ether is the main product of the reaction. This unwanted reaction outcome is suppressed by the addition of the MoCl₅.

Through addition of selenium dioxide as oxidizing agent, depending on the reaction conditions, 2,2′-biphenols or 2,2′-selenobiaryl ethers can be obtained as main products of the reaction. These reaction schemes are shown in FIG. 1. This is a scheme which shows aryls. However, an analogous scheme is also conceivable with phenols.

In earlier studies, it was found that the reaction can be shifted in the direction of the product desired in each case through addition of a base or an acid or a halogenated solvent (cf. applications of May 26, 2014). The reaction can be shifted in the direction of the 2,2′-selenobiaryl ether in a controlled manner by addition of a base having a pKb in the range from 8 to 11, whereas the reaction can be shifted in the direction of the 2,2′-biphenols in a controlled manner by addition of an acid having a pKa in the range from 0.0 to 5.0 or of a halogenated solvent.

### SUMMARY

It has now been found that the reaction can be shifted in the direction of the 2,2′-biphenol in a controlled manner by addition of a base and of a second oxidizing agent such as MoCl₅. Without addition of the second oxidizing agent, in contrast, a 2,2′-selenobiaryl ether would form. In the present case, the 2,2′-biphenol or 2,2′-biaryl is the desired main product, such that the unwanted reaction in the direction of the selenobiaryl ether is suppressed by the measures according to the invention.

Further advantages over the processes described in the prior art are that it is not necessary to work with exclusion of moisture or oxygen. This constitutes a distinct advantage over other synthesis routes. This direct method of C-C coupling is an efficient and selective process which stands out advantageously from the existing multistage synthesis routes.

Unconverted reactants and solvents used can be recovered by distillation and used for further reactions. Thus, the process according to the invention fulfils the requirements for an economic industrial scale process.

### BRIEF DESCRIPTION OF THE FIGURES

The foregoing and other features, aspects, and advantages of the present invention will become better understood with regard to the following description, appended claims, and accompanying drawings where:

FIG. 1 shows a reaction of aryls of the present invention; and

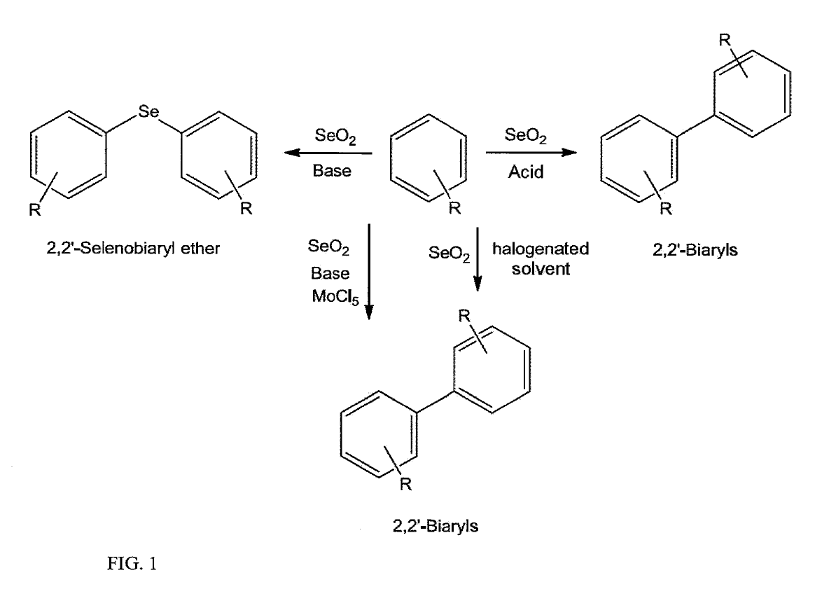

FIG. 2 is a reaction of biaryls scheme of the present invention.

### DESCRIPTION OF THE INVENTION

The invention therefore provides a process for preparing 2,2′-biaryls, in which a reaction mixture comprising a first aryl, a second aryl and selenium dioxide is heated, the first aryl being reacted with the second aryl to give the corresponding 2,2′-biaryl, wherein the reaction mixture additionally comprises molybdenum(V) chloride.

The sequence in which the two aryls, selenium dioxide and the MoCl₅ are added to the reaction mixture is unimportant. In principle, further components may also be present in the reaction mixture, for example solvent, acid or base.

Preferably, the first aryl and/or the second aryl is a compound of the general formula I:

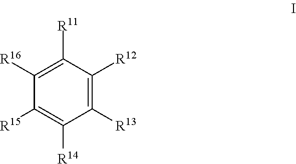

where R¹¹, R¹², R¹³, R¹⁴, R¹⁵, R¹⁶ are each independently selected from:
—H, —(C₁-C₁₂)-alkyl, —O—(C₁-C₁₂)-alkyl, —(C₆-C₂₀)-aryl, —O—(C₆-C₂₀)-aryl, —Cl, —F, —I, —OC═O— (C₁-C₁₂)-alkyl, —NH₂, —N[(C₁-C₁₂)-alkyl]₂;
where two adjacent radicals may but need not be joined to one another to give a fused system,
and where the alkyl and aryl groups mentioned may but need not be substituted, and at least one R¹¹, R¹², R¹³, R¹⁴, R¹⁵ or R¹⁶ radical is —H.

Further preferably, R¹¹, R¹², R¹³, R¹⁴, R¹⁵, R¹⁶ are each independently selected from:
—H, —(C₁-C₁₂)-alkyl, —O—(C₁-C₁₂)-alkyl, —(C₆-C₂₀)-aryl, —O—(C₆-C₂₀)-aryl, —Cl, —F, —I, where the alkyl and aryl groups mentioned may but need not be substituted, and at least one R¹¹, R¹², R¹³, R¹⁴, R¹⁵ or R¹⁶ radical is —H.

In a specific embodiment of the process, at least one
R¹¹, R¹², R¹³, R¹⁴, R¹⁵ or R¹⁶ radical is:
—O—(C₁-C₁₂)-alkyl.

In an even more specific embodiment of the process, R¹¹, R¹³, R¹⁴ are selected from:
—O—(C₁-C₁₂)-alkyl.

In the most specific embodiment of the process, R¹¹, R¹³, R¹⁴ are selected from:
—O—CH₃.

In one variant of the process, the first aryl corresponds to the second aryl, and so the two aryls are identical. This variant is thus a homo-coupling of two identical aryls. Ortho-ortho coupling thus gives rise to the desired 2,2′-biaryls.

In the present case, the 2,2′-biphenol is the desired main product, since biphenols are valuable units for synthesis of ligands for organometallic complex catalysts and other applications.

A particularly preferred embodiment of the invention accordingly serves for preparation of 2,2′-biphenols. The latter is consequently characterized in that the first aryl is a first phenol, in that the second aryl is a second phenol, and in that the first phenol is reacted with the second phenol to give the corresponding 2,2′-biphenol.

The first phenol and/or the second phenol is preferably a compound of the general formula II:

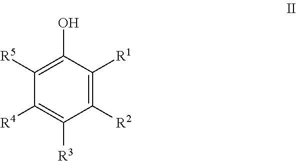

wherein R¹, R², R³, R⁴, R⁵ are each independently selected from:
—H, —(C₁-C₁₂)-alkyl, —O—(C₁-C₁₂)-alkyl, —(C₆-C₂₀)-aryl, —O—(C₆-C₂₀)-aryl, —Cl, —F, —I, —OC═O—(C₁-C₁₂)-alkyl,
where two adjacent radicals may but need not be joined to one another to give a fused system,
where the alkyl and aryl groups mentioned may but need not be substituted, and where at least R¹ or R⁵ is —H.

Preferably, R¹, R², R³, R⁴, R⁵ should each independently be selected from:
—H, —(C₁-C₁₂)-alkyl, —O—(C₁-C₁₂)-alkyl, —(C₆-C₂₀)-aryl, —O—(C₆-C₂₀)-aryl, —Cl, —F, —I,
where the alkyl and aryl groups mentioned may but need not be substituted, and where at least le or R⁵ is —H.

(C₁-C₁₂)-Alkyl and O—(C₁-C₁₂)-alkyl may each be unsubstituted or substituted by one or more identical or different radicals selected from:
(C₃-C₁₂)-cycloalkyl, (C₃-C₁₂)-heterocycloalkyl, (C₆-C₂₀)-aryl, fluorine, chlorine, cyano, formyl, acyl or alkoxycarbonyl.

(C₆-C₂₀)-Aryl and O—(C₆-C₂₀)-aryl may each be unsubstituted or substituted by one or more identical or different radicals selected from:
—H, —(C₁-C₁₂)-alkyl, —O—(C₁-C₁₂)-alkyl, —O—(C₆-C₂₀)-aryl, —(C₆-C₂₀)-aryl, -halogen (such as Cl, F, Br, I), —COO—(C₁-C₁₂)-alkyl, —CONH—(C₁-C₁₂)-alkyl, -(C₆-C₂₀)-aryl-CON[(C₁-C₁₂)-alkyl]₂, —CO—(C₁-C₁₂)-alkyl, —CO—(C₆-C₂₀)-aryl, —COOH, —OH, —SO₃H, —SO₃Na, —NO₂, —CN, —NH₂, —N[(C₁-C₁₂)-alkyl]₂.

In the context of the invention, the expression (C₁-C₁₂)-alkyl encompasses straight-chain and branched alkyl groups. Preferably, these groups are unsubstituted straight-chain or branched (C₁-C₈)-alkyl groups and most preferably (C₁-C₆)-alkyl groups. Examples of (C₁-C₁₂)-alkyl groups are especially methyl, ethyl, propyl, isopropyl, n-butyl, isobutyl, sec-butyl, tert-butyl, n-pentyl, 2-pentyl, 2-methylbutyl, 3-methylbutyl, 1,2-dimethylpropyl, 1,1-dimethylpropyl, 2,2-dimethylpropyl, 1-ethylpropyl, n-hexyl, 2-hexyl, 2-methylpentyl, 3-methylpentyl, 4-methylpentyl, 1,1-dimethylbutyl, 1,2-dimethylbutyl, 2,2-dimethylbutyl, 1,3-dimethylbutyl, 2,3-dimethylbutyl, 3,3 -dimethylbutyl, 1,1,2-trimethylpropyl, 1,2,2-trimethylpropyl, 1-ethylbutyl, 1-ethyl-2-methylpropyl, n-heptyl, 2-heptyl, 3-heptyl, 2-ethylpentyl, 1-propylbutyl, n-octyl, 2-ethylhexyl, 2-propylheptyl, nonyl, decyl.

The elucidations relating to the expression “—(C₁-C₁₂)-alkyl” also apply to the alkyl groups in —O—(C₁-C₁₂)-alkyl, i.e. in —(C₁-C₁₂)-alkoxy. Preferably, these groups are unsubstituted straight-chain or branched —(C₁-C₆)-alkoxy groups.

Substituted —(C₁-C₁₂)-alkyl groups and substituted —(C₁-C₁₂)-alkoxy groups may have one or more substituents, depending on their chain length. The substituents are preferably each independently selected from:
—(C₃-C₁₂)-cycloalkyl, —(C₃-C₁₂)-heterocycloalkyl, —(C₆-C₂₀)-aryl, fluorine, chlorine, cyano, formyl, acyl or alkoxycarbonyl.

In one variant of the process, the first phenol corresponds to the second phenol. The first phenol and the second phenol are therefore identical. This variant is thus a homo-coupling of two identical phenols. Ortho-ortho coupling thus gives rise to the desired 2,2′-biphenols.

In the context of the present studies, it has additionally been found that 2,2′-biaryls can be formed not only by coupling of the aryls but also by conversion of 2,2′-selenobiaryl ethers, but only in the presence of MoCl₅. Since the presence of the molybdenum(V) chloride is essential here too, and the same biaryls form, there is an underlying common inventive concept in this respect, namely the preparation of 2,2′-biaryls in the presence of molybdenum(V) chloride.

The invention therefore further provides a process for preparing 2,2′-biaryls in which a 2,2′-selenobiaryl ether is heated in the presence of molybdenum(V) chloride and converted to a 2,2′-biaryl.

The 2,2′-selenobiaryl ethers required had already been found in the course of prior studies; cf. DE 102014209974A1, likewise filed on May 26, 2014, and therefore as yet unpublished at the filing date.

To date, the 2,2′-selenobiaryl ethers have been regarded as useless by-products which had to be removed and disposed of in a costly and inconvenient manner. Due to the reaction route which has now been found, these 2,2′-selenobiaryl ethers can be used henceforth as reactants for the desired 2,2′-biaryls, and so they are no longer an unwanted by-product but instead a further product of value.

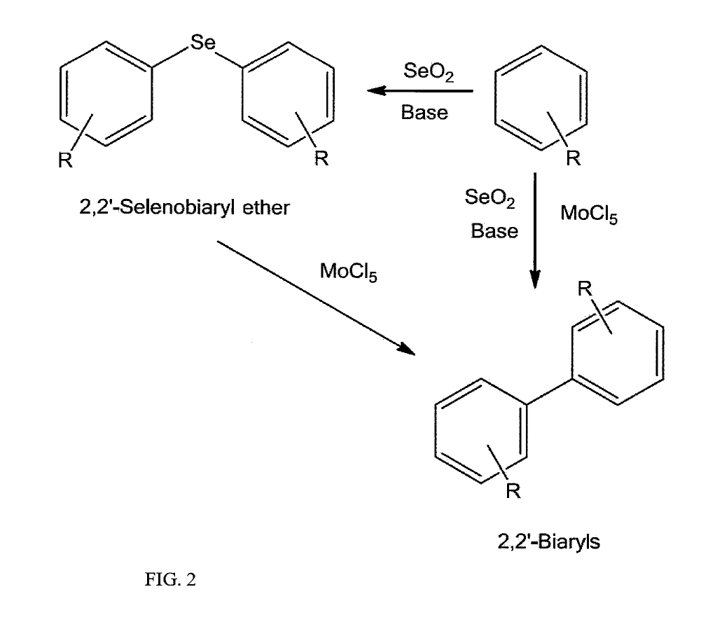

The addition of the MoCl₅ to 2,2′-selenobiaryl ethers allows the corresponding 2,2′-biaryls to be formed. The corresponding reaction scheme is shown in FIG. 2. Here too, for the sake of simplicity, only a scheme with aryls is shown. However, an analogous scheme applies to phenols.

It has been found that, proceeding from 2,2′-selenobiaryl ethers, the reaction can be shifted in the direction of the 2,2′-biaryl or 2,2′-biphenol in a controlled manner by addition of the MoCl₅. This is particularly advantageous since these compounds, compared to the corresponding 2,2′-selenobiaryl ethers, are the more interesting products having a multitude of fields of use on the industrial scale.

The 2,2′-selenobiaryl ether is preferably a compound of the general formula III:

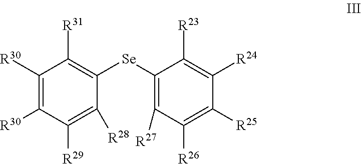

where R²³, R²⁴, R²⁵, R²⁶, R²⁷, R²⁸, R²⁹, R³⁰, R³¹ are each independently selected from:
—H, —(C₁-C₁₂)-alkyl, —O—(C₁-C₁₂)-alkyl, —(C₆-C₂₀)-aryl, —O—(C₆-C₂₀)-aryl, —Cl, —F, —I, —OC═O—(C₁-C₁₂)-alkyl, —NH₂, —N[(C₁-C₁₂)-alkyl]₂; OH;
where two adjacent radicals may but need not be joined to one another to give a fused system,
and where the alkyl and aryl groups mentioned may but need not be substituted.

Further preferably, R²³, R²⁴, R²⁵, R²⁶, R²⁷, R²⁸, R²⁹, R³⁰, R³¹ should each independently be selected from:
—H, —(C₁-C₁₂)-alkyl, —O—(C₁-C₁₂)-alkyl, —(C₆-C₂₀)-aryl, —O—(C₆-C₂₀)-aryl, —Cl, —F, —I, —OH.

Most preferably, R²³, R²⁴, R²⁵, R²⁶, R²⁷, R²⁸, R²⁹, R³⁰, R³¹ are each independently selected from: —H, —(C₁-C₁₂)-alkyl, —O—(C₁-C₁₂)-alkyl.

In one variant of the process, R²³, R²⁴, R²⁵, R²⁶, R²⁷, R²⁸, R²⁹, R³⁰, R³¹ are each independently selected from:
—H, —(C₁-C₁₂)-alkyl, —O—(C₁-C₁₂)-alkyl, OH.

In a specific variant of the process, R²³, R²⁴, R²⁵, R²⁶, R²⁷, R²⁸, R²⁹, R³⁰, R³¹ are each independently selected from:
—H, —(C₁-C₁₂)-alkyl, —O—(C₁-C₁₂)-alkyl.

In an even more specific embodiment of the process, R²³, R³¹ are selected from: —OH.

In the most specific embodiment, R²³, R³¹ are —OCH₃.

The reaction of the selenobiaryl ether to give the biaryl is effected at a temperature in the range from 50° C. to 110° C. Preference is given here to the range from 60° C. to 100° C., and particular preference to the range from 70° C. to 90° C.

Irrespective of whether the biaryls or biphenols are prepared from two aryls/phenols or from the 2,2′-selenobiaryl ether, the reaction should be conducted under the following boundary conditions:

The presence of a fluorinated solvent and/or of an acid should be avoided if possible. Both are possible on the laboratory scale, but impracticable on the industrial scale.

Nevertheless, the reaction can be conducted in the presence of a base in order to promote the reaction in the direction of the desired products. The base used should have a pKb in the range from 8 to 11. The base could be pyridine, quinoline, an amine base, for example triethylamine, or dimethylformamide. Particular preference is given to pyridine.

As well as the base, the reaction mixture may also comprise a solvent, for example tetrahydrofuran, ethylene glycol dimethyl ether, bis(2-methoxyethyl) ether, diethyl ether, toluene. All these solvents are free of fluorine and therefore of good availability. The purpose of the solvent is to assure good mixing and stirrability of the different components with one another.

Most preferably, the base is used as solvent. The base is thus simultaneously solvent; base and solvent are the same substance. This is permitted by pyridine, and for that reason the reaction is preferably conducted in the presence of pyridine.

A particular advantage of the reaction systems described here is that they are not susceptible to moist ambient air, i.e. a mixture of water vapor, oxygen and nitrogen, and in some cases even additionally liquid water. Consequently, there is no need to work with exclusion of air, which considerably simplifies the conduct of the reaction and makes it industrially practicable at all. The option of conducting the process in the presence of moist air is therefore of particular interest.

Preferably, the selenium dioxide is added in a molar ratio based on the sum total of the first and second aryls within a range from 0.25 to 1.2. Preference is given here to the range from 0.25 to 0.9, and particular preference to the range from 0.4 to 0.7.

The fact that the selenium dioxide can be used in a substoichiometric amount is a further advantage over the reaction described in the prior art with other inorganic oxidizing agents, for example AlCl₃, FeCl₃ or MnO₂. In principle, selenium dioxide should not be consumed in large amounts, and for that reason preference is given to the substoichiometric use thereof.

With regard to the amount of molybdenum(V) chloride to be added, the following figures can be given: 0.01 to 0.6 equivalent, preferably 0.05 to 0.2 equivalent, based on the reactant.

Preferably, the reaction is effected over a period in the range from 5 minutes to 24 hours. Preference is given here to the range from 15 minutes to 12 hours, and particular preference to the range from 15 minutes to 6 hours.

The invention is illustrated in detail hereinafter by working examples.

### Analysis

### NMR Spectroscopy

The NMR spectroscopy studies were conducted on multi-nucleus resonance spectrometers of the AC 300 or AV II 400 type from Bruker, Analytische Messtechnik, Karlsruhe. The solvent used was CDCl₃. The ¹H and ¹³C spectra were calibrated according to the residual content of undeuterated solvent using the NMR Solvent Data Chart from Cambridge Isotopes Laboratories, USA. Some of the ¹H and ¹³C signals were assigned with the aid of H,H-COSY, H,H-NOESY, H,C-HSQC and H,C-HMBC spectra. The chemical shifts are reported as δ values in ppm. For the multiplicities of the NMR signals, the following abbreviations were used: s (singlet), bs (broad singlet), d (doublet), t (triplet), q (quartet), m (multiplet), dd (doublet of doublets), dt (doublet of triplets), tq (triplet of quartets). All coupling constants J were reported in hertz (Hz) together with the number of bonds covered. The numbering given in the assignment of signals corresponds to the numbering shown in the formula schemes, which need not correspond to IUPAC nomenclature.

General Procedure (GP1)

The particular phenol or methoxyphenol was dissolved in the appropriate solvent (8.2 m). The reaction mixture was heated to 60° C. or 85° C. and selenium dioxide (0.6 equivalent) was added while stirring. The solvent was distilled under reduced pressure (temperature <70° C.). A frit was utilized for filtration of the solid constituents and filled for the purpose with 2.5 cm of silica gel (at the bottom) and 2.5 cm of zeolite (at the top). The distillation residue was taken up in the eluent and applied to the filtration column. Cyclohexane:acetate (95:5) was used to wash the product off the frit, and it was collected in fractions and freed of the eluent by distillation.

### Synthesis of the Selenobiaryl Ethers

### Bis(3,5-dimethyl-2-hydroxyphenyl)selenium (1)

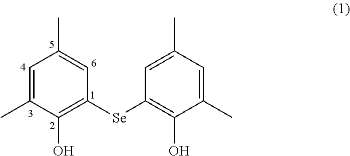

The reaction was conducted according to GP1 in a screw-top test tube. For this purpose, 2,4-dimethylphenol (1.00 g, 8.2 mmol) and selenium dioxide (0.54 g, 4.9 mmol) were dissolved in pyridine (1 ml, 8.2 m) and heated to 60° C. for 5 h. The product 1 (0.50 g, 3.1 mmol, 38%) was obtained as a colorless crystalline solid.

GC (hard, HP-5): t_{R}=14.2 min. ¹H NMR (400 MHz, CDCl₃): δ=7.12 (s, 2H, 6-H), 6.91 (s, 2H, 4-H), 5.97 (s,2H, OH), 2.23 (s, 6H, 3-CH₃) 2.23 (s, 6H, 5-CH₃) ppm. ¹³C NMR (100 MHz, CDCl₃): 151.7 (C-2), 133.2 (C-3), 133.1 (C-5), 130.4 (C-4), 124.2 (C-6), 114.9 (C-1), 20.3 (5-CH₃), 16.5 (3-CH₃) ppm. ⁷⁷Se NMR (76 MHz, CDCl₃): δ=163.36 ppm. MS (ESI⁺): m/z=345.04 [M+Na]⁺, 667.08 [2M+Na]⁺.

### Bis(2,3,5-trimethoxyphenyl)selenium (2)

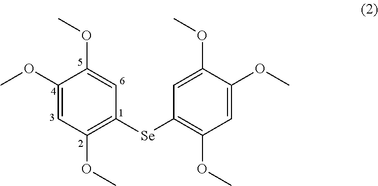

### 3,3′,5,5′-Tetramethylbiphenyl-2,2′-diol (3)

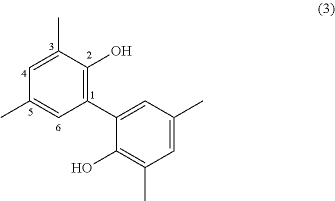

### Synthesis Proceeding From Phenol

The reaction was conducted according to GP1 in a screw-top test tube. For this purpose, 2,4-dimethylphenol (1.00 g, 8.2 mmol) and selenium dioxide (0.54 g, 4.9 mmol) were dissolved in formic acid (1 ml, 8.2 m) and heated to 70° C. for 2 h. The product 3 (0.61 g, 5.0 mmol, 61%) was obtained as a beige crystalline solid.

Reference in lab book (MMI-): 394, 395, 397, 400, 427, 443, 474.

### Synthesis Proceeding From Selenobiaryl Ether (1) (Inventive)

The selenobiaryl ether 1 (500 mg, 1.56 mmol) was dissolved in dichloromethane (10 ml). At 22° C., molybdenum pentachloride in dichloromethane (935 mg in 2 ml, 3.42 mmol) was added. To end the reaction, water (10 ml) was added after 20 min. The organic phase was removed and the solvent was distilled under reduced pressure. The distillation residue was taken up in the eluent and applied to the filtration column. Cyclohexane: acetate (95:5) was used to wash the product off the filtration column, and it was freed of the eluent by distillation. The product 3 was obtained in small amounts.

GC (hard, HP-5): t_{R}=12.3 min. R_{f}=0.2 (CH:EA 98:2). ¹H NMR (300 MHz, CDCl₃): δ=7.00 (s,2H, 6-H), 6.87 (s, 2H, 4-H), 5.07 (s,2H, OH), 2.27 (s, 12H, 3-CH₃, 5-CH₃) ppm. ¹³C NMR (75 MHz, CDCl₃): δ=149.2 (C-2), 132.1 (C-4), 130.0 (C-5), 128.5 (C-6), 125.1 (C-3), 122.1 (C-1), 20.4 (5-CH₃), 16.2 (3-CH₃) ppm. HRMS (ESI+) m/z for C₂₉H₃₄O₄Na [M+Na]⁺ calculated: 469.2355, found 469.2352.

### 1,2,4,1′,2′,4′-Hexamethoxy-5,5′-biphenyl (4) (Inventive)

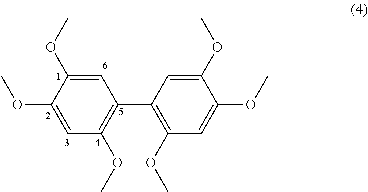

The selenobiaryl ether 2 (500 mg, 1.21 mmol) was dissolved in dichloromethane (10 ml). At 22° C., molybdenum pentachloride in dichloromethane (727 mg in 1.5 ml, 2.66 mmol) was added. To end the reaction, water (10 ml) was added after 20 min. The organic phase was removed and the solvent was distilled under reduced pressure. The distillation residue was taken up in the eluent and applied to a filtration column. Cyclohexane:acetate (95:5) was used to wash the product off the filtration column, and it was collected in fractions which were freed of the eluent by distillation. The product 4 (399 mg, 1.19 mmol, 99%) was obtained as a beige crystalline solid.

GC (hard, HP-5): t_{R}=15.3 min. ¹H NMR (400 MHz, CDCl₃): δ=6.82 (s, 2H), 6.63 (s, 2H), 3.93 (s, 6H), 3.84 (s, 6H), 3.76 (s, 6H) ppm. ¹³C NMR (101 MHz, CDCl₃): δ=151.38, 148.93, 143.02, 119.05, 115.45, 98.50, 57.04, 56.68, 56.25 ppm.

The results show that the process of the invention is a synthesis route by which 2,2′-biaryls can be prepared selectively and in a good yield proceeding from the 2,2′-selenobiaryl ethers.

It is striking that the substances used have not been dried in advance and the studies have not been conducted under inert conditions either. Normally—when working with exclusion of air—all solvents and solids would be dried beforehand and what are called Schlenk flasks would be employed. Schlenk flasks are reaction vessels which are first evacuated and then filled with argon or nitrogen until the air has been displaced, such that one is ultimately working only under protective gas (argon/nitrogen). This was not done in the present experiments. Consequently, all experiments were conducted in the presence of moist air, i.e. the ambient air in the laboratory. It is thus to be expected that, even in the case of production of the biaryls/biphenols in accordance with the invention on the ton scale, no exclusion of air will be necessary, which will achieve a considerable simplification of the process and reduction in costs.

What is claimed is:

1. A process for preparing 2,2′-biaryls, in which a reaction mixture comprising a first aryl, a second aryl and selenium dioxide is heated, the first aryl being reacted with the second aryl to give the corresponding 2,2′-biaryl, wherein the reaction mixture additionally comprises molybdenum(V) chloride.

2. The process according to claim 1, wherein the first aryl and/or the second aryl is a compound of the general formula I: 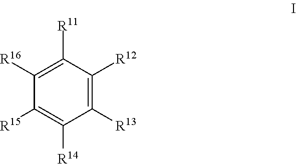
where R¹¹, R¹², R¹³, R¹⁴, R¹⁵, R¹⁶ are each independently selected from:
—H, —(C₁-C₁₂)-alkyl, —O—(C₁-C₁₂)-alkyl, —(C₆-C₂₀)-aryl, —O—(C₆-C₂₀)-aryl, —Cl, —F, —I, —OC═O—(C₁-C₁₂)-alkyl, —NH₂, —N[(C₁-C₁₂)-alkyl]₂;
where two adjacent radicals may but need not be joined to one another to give a fused system,
and where the alkyl and aryl groups mentioned may but need not be substituted, and at least one R¹¹, R¹², R¹³, R¹⁴, R¹⁵ or R¹⁶ radical is —H.

3. The process according to claim 2, wherein R¹¹, R¹², R¹³, R¹⁴, R¹⁵, R¹⁶ are each independently selected from:
—H, —(C₁-C₁₂)-alkyl, —O—(C₁-C₁₂)-alkyl, —(C₆-C₂₀)-aryl, —O—(C₆-C₂₀)-aryl, —Cl, —F, —I,
where the alkyl and aryl groups mentioned may but need not be substituted,
and at least one R¹¹, R¹², R¹³, R¹⁴, R¹⁵ or R¹⁶ radical is —H.

4. The process according to claim 2, wherein the first aryl and the second aryl are identical.

5. The process according to claim 1 for preparing 2,2′-biphenols, wherein the first aryl is a first phenol, in that the second aryl is a second phenol, and in that the first phenol is reacted with the second phenol to give the corresponding 2,2′-biphenol.

6. The process according to claim 5, wherein the first phenol and/or the second phenol is a compound of the general formula II: 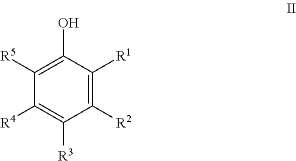
wherein R¹, R², R³, R⁴, R⁵ are each independently selected from:
—H, —(C₁-C₁₂)-alkyl, —O—(C₁-C₁₂)-alkyl, —(C₆-C₂₀)-aryl, —O—(C₆-C₂₀)-aryl, —Cl, —F, —I, —OC═O—(C₁-C₁₂)-alkyl,
where two adjacent radicals may but need not be joined to one another to give a fused system,
where the alkyl and aryl groups mentioned may but need not be substituted,
and where at least le or R⁵ is —H.

7. The process according to claim 6, where
R¹, R², R³, R⁴, R⁵ are each independently selected from:
—H, —(C₁-C₁₂)-alkyl, —O—(C₁-C₁₂)-alkyl, —(C₆-C₂₀)-aryl, —O—(C₆-C₂₀)-aryl, —Cl, —F, —I,
where the alkyl and aryl groups mentioned may but need not be substituted,
and where at least le or R⁵ is —H.

8. The process according to claim 6, wherein the first phenol and the second phenol are identical.

9. A process for preparing 2,2′-biaryls, wherein a 2,2′-selenobiaryl ether is heated in the presence of molybdenum(V) chloride and converted to a 2,2′-biaryl.

10. The process according to claim 1,
wherein the 2,2′-selenobiaryl ether is a compound of the general formula III: 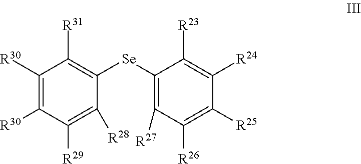
where R²³, R²⁴, R²⁵, R²⁶, R²⁷, R²⁸, R²⁹, R³⁰, R³¹ are each independently selected from:
—H, —(C₁-C₁₂)-alkyl, —O—(C₁-C₁₂)-alkyl, —(C₆-C₂₀)-aryl, —O—(C₆-C₂₀)-aryl, —Cl, —F, —I, —OC═O—(C₁-C₁₂)-alkyl, —NH₂, —N[(C₁-C₁₂)-alkyl]₂; OH;
where two adjacent radicals may but need not be joined to one another to give a fused system,
and where the alkyl and aryl groups mentioned may but need not be substituted.

11. The process according to claim 10,
where R²³, R²⁴, R²⁵, R²⁶, R²⁷, R²⁸, R²⁹, R³⁰, R³¹ are each independently selected from:
—H, —(C₁-C₁₂)-alkyl, —(C₆-C₂₀)-aryl, O—(C₆-C₂₀)-aryl, —Cl, —F, —I, —OH.

12. The process according to claim 10,
where R²³, R²⁴, R²⁵, R²⁶, R²⁷, R²⁸, R²⁹, R³⁰, R³¹ are each independently selected from: —H, —(C₁-C₁₂)-alkyl, —O—(C₁-C₁₂)-alkyl.

13. The process according to claim 1, conducted in the absence of a fluorinated solvent and/or in the absence of an acid.

14. The process according to claim 1, conducted in the presence of a base selected from pyridine, quinoline, amine bases, dimethylformamide, and/or in the presence of a solvent selected from tetrahydrofuran, ethylene glycol dimethyl ether, bis(2-methoxyethyl) ether, diethyl ether, toluene.

15. The process according to claim 1, conducted in the presence of moist air.
